(19) United States
(12) Patent Application Publication
(10) Pub. No.: US 2006/0140898 A1
(43) Pub. Date: Jun. 29, 2006

(54) Use of a combination of at least one associated polymer and at least one amphiphilic diblock copolymer for thickening cosmetic compositions

(76) Inventors: Claude Dubief, le Chesnay (FR); Luc Nicolas Morgantini, Rully (FR); Anne-Laure Bernard, Singapore (SG); Frederic Simonet, Touqin (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/283,868

(22) Filed: Nov. 22, 2005

Related U.S. Application Data
(60) Provisional application No. 60/646,612, filed on Jan. 26, 2005.

(30) Foreign Application Priority Data
Nov. 22, 2004 (FR) 04 12382

(51) Int. Cl. A61K 8/81; A61K 8/73

(52) U.S. Cl. 424/70.13; 424/70.15; 424/70.17

(57) ABSTRACT

The present disclosure relates to cosmetic compositions comprising, in a physiologically acceptable aqueous medium, a combination of
at least one associative polymer comprising at least one hydrophilic part and at least one C₈₋₄₀ fatty chain, and at least one linear diblock block copolymer composed of a hydrophobic block and a hydrophilic block, with the proviso that the at least one linear diblock block copolymer is not chosen from block copolymers of ethylene oxide and propylene oxide, block copolymers with urethane units, and block copolymers with siloxane units. The present disclosure also relates to the use of such a combination for thickening or gelling aqueous cosmetic compositions.

This application claims benefit of U.S. Provisional Application No. 60/646,612, filed Jan. 26, 2005, the contents of which are incorporated herein by reference. This application also claims benefit of priority under 35 U.S.C. §119 to French Patent Application No. 04 12382, filed Nov. 22, 2004, the contents of which are also incorporated by reference.

The present disclosure relates to the use of a combination of at least one associative polymer comprising at least one hydrophilic part and at least two fatty chains, and of at least one linear diblock copolymer for thickening aqueous cosmetic compositions, and also to aqueous cosmetic compositions thickened by such a combination.

The thickening and/or gelling of aqueous media by polymers has been an important research topic for a long time, for example, in the field of cosmetology and of pharmacy. Obtaining a beneficial thickening effect by a water-soluble polymer generally presumes a high molar mass and a substantial hydrodynamic volume, and the gelling of an aqueous medium requires the formation of a three-dimensional polymeric network. Using very high molar mass polymers of this kind, however, may give rise to a certain number of problems, such as the fairly unpleasant texture and the difficulty of spreading of the gels obtained.

One beneficial approach has been to use, as thickeners, polymers which are capable, in solution, of undergoing reversible association with one another or with other molecules or particles. This physical association can give rise to thixotropic or shear-thinning macromolecular systems, i.e. systems whose viscosity is dependent on the shearing forces to which they are subjected. The forces of interaction involved may differ greatly in kind: such as, for example, electrostatic forces, hydrogen bond forces or hydrophobic interaction forces.

Polymers capable of undergoing reversible association with themselves or with other molecules by hydrophobic interaction are termed “associative polymers.” These are polymers comprising at least one hydrophilic moiety, making them at least partly soluble in water, and at least one hydrophobic part, via which the polymers interact and associate with one another or with other molecules.

Associative polymers are commonly used to thicken or gel aqueous media. It is also known that the thickening power of these associative polymers may be reinforced by simultaneous use of surfactants. It is generally acknowledged that, in this case, the hydrophobic chains of the associative polymers participate in the formation of mixed micelles (surfactants+hydrophobic part of the associative polymer), which constitute the nodes of a three-dimensional polymeric network.

### BRIEF DESCRIPTION OF THE DRAWING

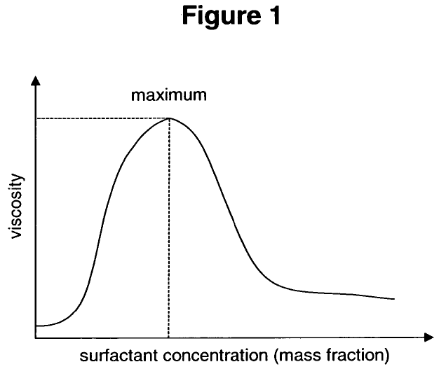

The existence of mixed micelles (surfactants+hydrophobic part of the associative polymer) can be demonstrated by the existence of a maximum in the plot of viscosity as a function of surfactant concentration, for a given concentration of associative polymer as shown in FIG. 1 attached. FIG. 1 of the present disclosure is a “bell curve,” from which it is possible to calculate the thickening factor, which is defined as the ratio between the viscosity at the (local) maximum of the plot and the viscosity of the solution of associative polymer alone at the concentration used. The value on the abscissa of the maximum of “bell curves” of this kind characterizes the stoichiometry of the maximum interaction between the surfactants and the hydrophobic parts of the associative polymer, and the value on the ordinate gives the “maximum thickening factor,” which characterizes the thickening capacity of the combination.

However, not all surfactants may result in a reinforced thickening effect of this kind. Others may give rise to problems of ineffectiveness in cosmetic compositions, and it may then be desirable to replace them. Finally, a certain number of thickening systems of this kind, based on surfactants and associative polymers, may be extremely sensitive to the presence of salts and may not, therefore, allow the viscosity to be controlled satisfactorily.

In the course of research into new polymeric thickening systems for aqueous media, the Inventors discovered, surprisingly, that certain amphiphilic block copolymers were successful at reinforcing the thickening power of associative polymers and could be used, consequently, to replace in part or in whole the typical surfactants exhibiting the above disadvantages.

The present disclosure accordingly relates to a method for thickening or gelling aqueous cosmetic compositions comprising, adding to the compositions:

at least one associative polymer comprising at least one hydrophilic part and at least one C₈₋₄₀ fatty chain, and

at least one linear diblock block copolymer comprising a hydrophobic block and a hydrophilic block, with the proviso that the at least one linear diblock block copolymer does not comprise block copolymers of ethylene oxide and propylene oxide, block copolymers with urethane units, and block copolymers with siloxane units.

The present disclosure also relates to cosmetic compositions comprising, in a physiologically acceptable aqueous medium, a combination of

at least one associative polymer comprising at least one hydrophilic part and at least one C₈₋₄₀ fatty chain, and

at least one linear diblock block copolymer comprising a hydrophobic block and a hydrophilic block, with the proviso that the at least one linear diblock copolymer is not chosen from block copolymers of ethylene oxide and propylene oxide, block copolymers with urethane units, and block copolymers with siloxane units.

As used here, an “aqueous medium” is understood to mean a solvent medium which is liquid at ambient temperature and atmospheric pressure and comprises a substantial amount of water, for example, an amount greater than or equal to 30% by weight, for instance, greater than or equal to 50% by weight, and such as greater than or equal to 75% by weight, relative to the total weight of the solvent medium, the remainder of the medium being composed of at least one water-miscible, physiologically acceptable organic solvent, such as lower C₁-C₄ alcohols, for instance, ethanol, isopropanol, tert-butanol and n-butanol, or alkylene glycols such as propylene glycol and glycerol.

Similarly, as used herein, the term “aqueous compositions” is understood to mean compositions comprising an aqueous medium as defined above.

The linear diblock block copolymers used according to the present disclosure are “amphiphilic” copolymers: that is, copolymers comprising both a hydrophobic block and a hydrophilic block.

As used herein, the term “hydrophobic block” is understood to mean a block comprising at least 75 mol % of water-insoluble monomers, and the term “hydrophilic block” is understood to mean a block comprising at least 75 mol % of water-soluble monomers.

The water-soluble monomers forming the hydrophilic block of the diblock copolymers used according to the present disclosure can be chosen from anionic, nonionic and cationic monomers, and may be used alone or in the form of a mixture comprising at least two different monomers.

As used herein, the phrase “monomers or polymers which are soluble” is understood to mean monomers or polymers in a given medium, which, when introduced into the medium at 25° C. and at a concentration by weight of 0.5%, in neutralized form if necessary, allow a macroscopically homogeneous and transparent solution to be obtained, in other words a solution whose light transmittance at a wavelength of 500 nm through a sample with a thickness of 1 cm is greater than or equal to 70%, such as greater than or equal to 80%.

Non-limiting examples of anionic water-soluble monomers include ethylenically unsaturated carboxylic acids, such as acrylic acid, methacrylic acid, itaconic acid, fumaric acid, crotonic acid, and maleic acid or anhydride, 2-acrylamido-2-methylpropanesulphonic acid, styrenesulphonic acid, vinylsulphonic acid and vinylphosphonic acid.

Non-limiting examples of nonionic water-soluble monomers include, among others, acrylamide, N—C₁₋₆ alkyl-acrylamides, N,N-di-C₁₋₃ alkyl-acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol (copolymerized in the form of vinyl acetate and then hydrolyzed), ethylene oxide, hydroxyethyl acrylate, hydroxypropyl acrylate, hydroxyethyl methacrylate, and hydroxypropyl methacrylate.

Among the cationic water-soluble monomers that may be used, non-limiting mention may be made of, for example, dimethyldiallylammonium chloride, methylvinylimidazolium chloride, 2-vinylpyridine, 4-vinylpyridine, N-(C₁₋₄ alkyl)-4-vinylpyridinium halides such as N-methyl-4-vinylpyridinium iodide, 2-methyl-5-vinylpyridine, vinylamine, and monomers of formula
H₂C═CR¹—CO—X²
wherein

R¹ is chosen from a hydrogen atom and methyl groups,

X² is chosen from linear and branched C₁₋₆ hydrocarbon groups which carry at least one amine functional group chosen from primary, secondary and tertiary amine functional groups; or alternatively carry at least one quaternary nitrogen atom; or alternatively carry a group of formula NHR² or of formula NR²R³, in which R² and R³, which may be identical or different, are chosen from linear and branched C₁₋₆ hydrocarbon groups which carry at least one amine functional group chosen from primary, secondary and tertiary amine functional groups, or alternatively, at least one quaternary nitrogen atom.

The water-insoluble monomers forming the hydrophobic block of the diblock copolymers can be chosen from, for example vinylaromatic monomers such as styrene and its alkyl derivatives, such as 4-butylstyrene, α-methylstyrene and vinyltoluene; dienes such as butadiene and 1,3-hexadiene; alkyl derivatives of dienes; such as isoprene and dimethylbutadiene; chloroprene; C₁₋₁₀ alkyl acrylates; C₆₋₁₀ aryl acrylates; C₆₋₁₀ aralkyl acrylates; C₁₋₁₀ alkyl methacrylates; C₆₋₁₀ aryl methacrylates; C₆₋₁₀ aralkyl methacrylates, such as, for example methyl, ethyl, n-butyl, 2-ethylhexyl, tert-butyl, isobornyl, phenyl and benzyl (meth)acrylates; vinyl acetate; vinyl ethers of formula CH₂═CH—O—R and allyl ethers of formula CH₂═CH—CH₂—O—R in which R is chosen from C₁₋₆ alkyl groups; acrylonitrile; vinyl chloride; vinylidene chloride; caprolactone; ethylene; propylene; fluorinated vinyl monomers; vinyl monomers comprising a perfluorinated chain, such as fluoroalkyl acrylates, fluoroalkyl methacrylates, and alkyl α-fluoroacrylates.

As discussed above with regard to the definition of the hydrophobic and hydrophilic blocks of the diblock copolymers, the water-insoluble monomers and the water-soluble monomers can be present in their respective blocks in an amount greater than or equal to 75 mol %. Expressed alternatively, the at least one hydrophobic block can comprise less than or equal to 25 mol % of at least one water-soluble monomer. For example, the at least one hydrophobic block can comprise less than or equal to 10 mol %, such as less than or equal to 5 mol %, of at least one water-soluble monomer.

Similarly, the at least one hydrophilic block may comprise less than or equal to 25 mol %, for instance less than or equal to 10 mol %, such as less than 5 mol %, of at least one water-insoluble monomer.

The at least one linear diblock copolymer as disclosed herein may, of course, also be chosen from those copolymers in which the at least one hydrophilic block and the at least one hydrophobic block are composed exclusively of water-soluble monomers and of water-insoluble monomers, respectively. These blocks may be homopolymer blocks or copolymer blocks comprising at least two different monomers of the same type.

The number-average molecular mass of each block, whether it be hydrophobic or hydrophilic, copolymeric or homopolymeric, can range from 500 to 100,000, such as from 500 to 50,000, with a polydispersity index (M_{w}/Mₙ) ranging from 1.01 to 3.0, for instance, from 1.1 to 2.5.

The weight ratio of the at least one hydrophobic block to the at least one hydrophilic block of the block copolymer can range, for example, from 1/20 to 20/1, such as from 1/10 to 10/1.

In order to reinforce effectively the thickening power of the associative polymers described in detail above, the at least one diblock block copolymer can be at least partially soluble and dissolved in the aqueous medium used.

The Inventors have obtained beneficial results with anionic diblock block copolymers comprising a hydrophilic block formed from anionic water-soluble monomers, or formed from a mixture of anionic and nonionic water-soluble monomers.

In one embodiment of the present disclosure the at least one diblock block copolymer comprise a hydrophobic homopolymer block and an anionic hydrophilic homopolymer block.

Examples of polymers of this kind comprising two homopolymer blocks include polystyrene-poly(sodium acrylate) copolymers and polystyrene-poly(sodium styrene sulphonate) copolymers.

The viscosity of the compositions of the present disclosure, for example, can range from 100 m·Pa·s to 10⁶ m·Pa·s, at a temperature of 25° C. and a shear rate of 1 s⁻¹. It can be measured by means of a Haake RS 600 instrument.

The amount of the at least one diblock block copolymer used in the present disclosure depends, of course, on the nature and amount of the at least one associative polymer used, on the thickening factor of the diblock copolymer/associative polymer pairing, and, for instance, on the desired viscosity. The cosmetic compositions of the present disclosure can comprise diblock block copolymer concentrations ranging from 0.001% to 20% by weight, for instance, from 0.005% to 10% by weight, such as from 0.01 and 5%, relative to the total weight of the composition.

For the present disclosure, it is possible, in principle, to use any type of known associative polymer as defined above. Thus, the at least one associative polymer can be chosen from anionic, cationic, amphoteric and nonionic polymers. In one embodiment of the present disclosure, the at least one associative polymer is chosen from cationic, anionic and nonionic polymers.

In still another embodiment of the present disclosure, cationic associative polymers are used.

The at least one associative polymer can be present in the composition according to the present disclosure in an amount ranging from 0.01% to 20% by weight, relative to the total weight of the composition, for example from 0.05% and 10%, such as from 0.1% and 5% by weight, relative to the total weight of the composition.

In one embodiment of the present disclosure, the weight ratio of the at least one associative polymer)/at least one diblock polymer can range from 0.01 to 100, for instance, from 1 to 50, and such as from 5 to 30.

Non-limiting examples of anionic associative polymers that may be used include the following polymers:

(I) Polymers comprising at least one hydrophilic unit and at least one fatty-chain allyl ether unit, for example those whose at least one hydrophilic unit is composed of an ethylenic unsaturated anionic monomer, for instance of a vinyl carboxylic acid, such as of an acrylic acid or a methacrylic acid or mixtures thereof, and whose at least one fatty-chain allyl ether unit is a C₈₋₃₀ fatty alcohol allyl ether of formula (I):
CH₂═CR′—CH₂—OBₙ—R (I)

wherein R′ is chosen from an H atom and CH₃ groups, B is an ethyleneoxy radical, n is an integer ranging from 0 to 100, R is a hydrocarbon radical chosen from alkyl, arylalkyl, aryl, alkylaryl and cycloalkyl radicals comprising from 8 to 30 carbon atoms, for instance from 10 to 24, and such as from 12 to 18 carbon atoms. In one embodiment of the present disclosure, the at least one fatty-chain allyl ether unit of formula (I) is a unit in which R′ is H, n is 10, and R is a stearyl (C₁₈) radical.

Anionic associative polymers of this type and their preparation by emulsion polymerization are described in European Patent Application No. EP-0 216 479.

Among these anionic associative polymers, mention may be made of polymers formed from 20% to 60% by weight of acrylic acid and/or methacrylic acid, 5% to 60% by weight of lower alkyl (meth)acrylates, 2% to 50% by weight of fafty-chain allyl ether of formula (I), and 0% to 1% by weight of a crosslinking agent which is a copolymerizable polyethylenic unsaturated monomer, such as diallyl phthalate, allyl (meth)acrylate, divinylbenzene, (poly)ethylene glycol dimethacrylate and methylenebisacrylamide.

In one embodiment of the present disclosure, the crosslinking agent is chosen from crosslinked terpolymers of methacrylic acid, of ethyl acrylate and of polyethylene glycol (10 EO) stearyl alcohol ether (Steareth 10), for instance those sold by Allied Colloids under the names Salcare SC80® and Salcare SC90®, which are aqueous 30% emulsions of a crosslinked terpolymer of methacrylique acid, ethyl acrylate and steareth-10 allyl ether (40/50/10).

(II) Polymers comprising at least one hydrophilic unit of olefinically unsaturated carboxylic acid type and at least one hydrophobic unit of C₁₀-C₃₀ alkyl unsaturated carboxylate type.

In one embodiment, for example, these polymers are chosen from those in which the at least one hydrophilic unit of unsaturated carboxylic acid type is chosen from the monomers of formula (II): 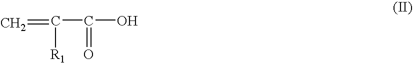
wherein R₁ is chosen from an H atom and CH₃ and C₂H₅ groups, (i.e., acrylic acid, methacrylic acid, and ethacrylic acid units), and in which the at least one hydrophobic unit of C₁₀-C₃₀ alkyl unsaturated carboxylate type is chosen from those of formula (III): 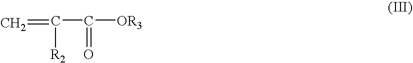
wherein R₂ is chosen from an H atom and CH₃ and C₂H₅ groups, for example chosen from H and CH₃, and R₃ is a C₁₀-C₃₀ group, such as a C₁₂-C₂₂, alkyl radical.

C₁₀-C₃₀ alkyl unsaturated carboxylates in accordance with the present disclosure include, for example, lauryl acrylate, stearyl acrylate, decyl acrylate, isodecyl acrylate, dodecyl acrylate, and the corresponding methacrylates: lauryl methacrylate, stearyl methacrylate, decyl methacrylate, isodecyl methacrylate and dodecyl methacrylate.

Anionic associative polymers of this type and their preparation are described in U.S. Pat. Nos. 3,915,921 and 4,509,949.

Among this type of anionic associative polymers, mention may be made, for example, of polymers synthesized from a monomer mixture comprising acrylic acid, an ester of formula (III) above wherein R₂ is chosen from an H atom and CH₃ groups and R₃ is an alkyl radical comprising 12 to 22 carbon atoms, and a crosslinking agent which is a copolymerizable polyethylenic unsaturated monomer, such as diallyl phthalate, allyl (meth)acrylate, divinylbenzene, (poly)ethylene glycol dimethacrylate and methylenebisacrylamide.

Further among these anionic associative polymers, mention may be made, for instance, of those composed of 95% to 60% by weight of acrylic acid (hydrophilic unit), 4% to 40% by weight of C₁₀-C₃₀ alkyl acrylate (hydrophobic unit), and 0% to 6% by weight of crosslinking polymerizable monomer, as well as those composed of 98% to 96% by weight of acrylic acid (hydrophilic unit), 1% to 4% by weight of C₁₀-C₃₀ alkyl acrylate (hydrophobic unit), and 0.1% to 0.6% by weight of crosslinking polymerizable monomer such as those described above.

In one embodiment of the present disclosure the polymers used are the products sold by Goodrich under the trade names Pemulen TR1®, Pemulen TR2®, Carbopol 1382®, and the product sold by SEPPIC under the name Coatex SX®.

(III) Maleic anhydride/C₃₀-C₃₈ α-olefin/alkyl maleate terpolymers such as the product (maleic anhydride/C₃₀-C₃₈ α-olefin/isopropyl maleate copolymer) sold under the name Performa V 1608® by Newphase Technologies.

(IV) Acrylic terpolymers comprising from 20% to 70% by weight of an α,β-unsaturated carboxylic acid, from 20% to 80% by weight of a non-surfactant α,β-unsaturated monomer other than the aforementioned acid, and from 0.5% to 60% by weight of a nonionic surfactant monomer which is the product of reaction of a monohydric surfactant with an ethylenically unsaturated monoisocyanate, such as those described European Patent Application No. EP-A-0 173 109, and for example, a methacrylic acid/methyl acrylate/ethoxylated (40 EO) behenyl alcohol dimethyl-meta-isopropenylbenzyl isocyanate terpolymer in aqueous 25% dispersion, sold under the name Viscophobe DB 1000® by Amerchol.

(V) Copolymers including among their monomers an α,β-monoethylenically unsaturated carboxylic acid and an ester of an α,β-monoethylenically unsaturated carboxylic acid and an alkoxylated fatty alcohol.

For example, these compounds may also include as a monomer an ester of an α,β-monoethylenically unsaturated carboxylic acid and a C₁-C₄ alcohol. One example of this type of compound is Aculyn 22® sold by Rohm & Haas, which is a methacrylic acid/ethyl acrylate/alkoxylated stearyl methacrylate terpolymer.

Among the cationic associative polymers that can be used in the present disclosure, non-limiting mention may be made of, for example:

(I) Cationic associative polyurethanes of the class described in French Patent Application Publication No. FR-A-2 811 993. These cationic associative polyurethanes can be chosen from those of general formula (Ia):
R—X—(P)ₙ—[L—(Y)ₘ]ᵣ—L′—(P′)ₚ—X′—R′ (Ia)
wherein:

R and R′, which may be identical or different, can be chosen from hydrophobic groups and a hydrogen atoms;

X and X′, which may be identical or different, can be chosen from groups comprising at least one amine functional group, optionally carrying a hydrophobic group; and L″ groups;

L, L′ and L″, which may be identical or different, are chosen from groups derived from a diisocyanate;

P and P′, which may be identical or different, are chosen from groups comprising at least one amine functional group optionally carrying a hydrophobic group;

Y is a hydrophilic group;

r is an integer ranging from 1 to 100, for instance from 1 to 50, and such as from 1 to 25,

n, m and p, which may be identical or different, can range from 0 to 1,000,

wherein the polyurethanes of formula (Ia) comprise at least one protonated or quaternized amine functional group and at least one hydrophobic group.

In one embodiment of the present disclosure, in the polyurethanes, the only hydrophobic groups are the groups R and R′ at the chain ends.

Mention may be made of a class of cationic associated polyurethanes that corresponds those of formula (Ia) described above wherein R and R′, which may be identical or different, are chosen from hydrophobic groups; X and X′ each represent a group L″; n and p range from 1 to 1,000; and L, L′, L″, P, P′, Y and m are as defined above.

Further mention may also be made of a class of cationic associated polyurethanes that corresponds to those of formula (Ia) above wherein R and R′, which may be identical or different, are chosen from hydrophobic groups; X and X′, which may be identical or different, are chosen from L″ groups; n and p are 0; and L, L′, L″, Y and m are as defined above. The fact that n and p are zero means that in this instance, these polymers do not comprise units derived from an amine-functional monomer incorporated into the polymer during the polycondensation. The protonated amine functional groups of these polyurethanes result from the hydrolysis of excess isocyanate functional groups at the chain end, followed by alkylation of the primary amine functional groups formed with alkylating agents having a hydrophobic group, i.e. compounds of type RQ or R′Q, wherein R and R′ are as defined above and Q is chosen from leaving groups, such as halides, sulphates, etc.

Yet still further mention may be made of a class of cationic associative polyurethanes that corresponds to those of formula (Ia) above wherein R and R′, which may be identical or different, are chosen from hydrophobic groups; X and X′, which may be identical or different, are chosen from groups comprising a quaternary amine; n and p are zero; and L, L′, Y and m are as defined above.

When X and/or X′ are chosen from groups comprising a tertiary or quaternary amine, X and/or X′ can be chosen from at least one of the following formulae: 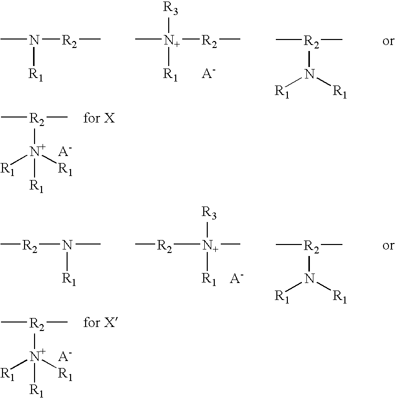
wherein:

R₂ is chosen from linear and branched alkylene radicals comprising from 1 to 20 carbon atoms and optionally comprising a group chosen from saturated or unsaturated rings, or an arylene radical, it being possible for at least one of the carbon atoms to be replaced by at least one heteroatom chosen from N, S, O and P;

R₁ and R₃, which may be identical or different, are chosen from linear and branched C₁-C₃₀ alkyl and alkenyl radicals, and aryl radicals, it being possible for at least one of the carbon atoms to be replaced by at least one heteroatom chosen from N, S, O and P; and

A⁻is a physiologically acceptable counterion.

The groups L, L′ and L″, which may be identical or different are chosen from those of formula: 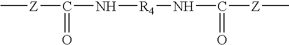
wherein:

Z is chosen from —O— and —S— atoms, and —NH— groups, and

R₄ is chosen from linear and branched alkylene radicals comprising from 1 to 20 carbon atoms and optionally comprising a group chosen from a saturated or unsaturated ring, or an arylene radical, it being possible for at least one of the carbon atoms to be replaced by at least one heteroatom chosen from N, S, O and P.

The groups P and P′ comprising an amine functional group can be chosen from at least one of the following formulae: 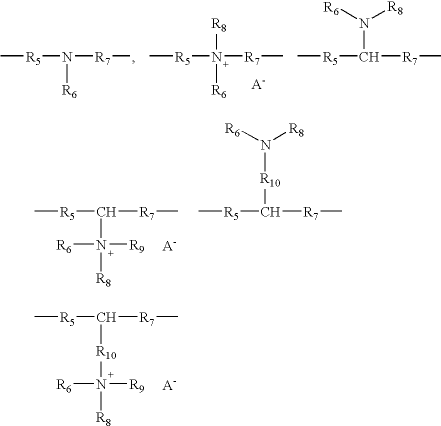
wherein:

R₅ and R₇ have the same definitions as R₂, defined above,

R₆, R₈ and R₉ have the same definitions as R₁ and R₃, defined above,

R₁₀ is chosen from linear and branched alkylene groups which are optionally unsaturated and may comprise at least one heteroatom chosen from N, O, S and P, and

A⁻is a physiologically acceptable counterion.

With regard to the definition of Y, a hydrophilic group is understood to be a polymeric or non-polymeric water-soluble group.

By way of non-limiting example, when Y is not a polymer, mention may be made of ethylene glycol, diethylene glycol and propylene glycol.

When Y is a hydrophilic polymer, in accordance with one embodiment of the present disclosure, mention may be made, by way of example, of polyethers, sulphonated polyesters, sulphonated polyamides or a mixture of these polymers. For example, the hydrophilic compound may be a polyether, such as a poly(ethylene oxide) or poly(propylene oxide).

The cationic associative polyurethanes of formula (Ia) are formed from diisocyanates and various compounds possessing functional groups comprising labile hydrogen. The functional groups comprising labile hydrogen may be alcohol functional groups, primary and secondary amine functional groups, and thiol functional groups, giving, after reaction with the diisocyanate functional groups, polyurethanes, polyureas and polythioureas, respectively. As used herein, the term “polyurethanes” is understood to mean these three types of polymer, namely polyurethanes proper, polyureas and polythioureas, and also copolymers of thereof.

The hydrophobic group of the polyurethane of formula (Ia) may also result from the quaternization reaction of the tertiary amine of the compound comprising at least one tertiary amine unit. Thus the hydrophobic group is introduced by the quaternizing agent. This quaternizing agent is a compound of type RQ or R′Q in which R and R′ are as defined above and Q is a leaving group such as a halide, a sulphate, etc.

The hydrophilic group, labelled Y, in the formula (Ia) is optional. This is because the units comprising a quaternary or protonated amine functional group may be sufficient to provide the necessary solubility or water-dispersibility for this type of polymer in an aqueous solution. Although the presence of a hydrophilic group Y is optional, in one embodiment of the present disclosure, the cationic associative polyurethanes are chosen from those which include such a group.

(II) Quaternized, fatty-chain derivatives of celluloses or hydroxyethylcelluloses, chosen, for example, from the following:

quaternized celluloses modified by groups comprising at least one fatty chain, such as alkyl, arylalkyl and alkylaryl groups comprising from 8 to 30 carbon atoms, or mixtures thereof,

quaternized hydroxyethylcelluloses modified with groups comprising at least one fatty chain, such as alkyl, arylalkyl and alkylaryl groups comprising from 8 to 30 carbon atoms, or mixtures thereof.

In one embodiment of the present disclosure, the aryl radicals are chosen from phenyl, benzyl, naphthyl and anthryl groups.

Non-limiting examples that may be mentioned of quaternized alkylhydroxyethyl celluloses comprising C₈-C₃₀ fatty chains are the products Quatrisoft LM 200®, Quatrisoft LM-X 529-18-A®, Quatrisoft LM-X 529-18B® (C₁₂ alkyl), and Quatrisoft LM-X 529-8® (C18 alkyl), sold by Amerchol, and the products Crodacel QM®, Crodacel QL® (C₁₂ alkyl), and Crodacel QS® (C₁₈ alkyl), sold by Croda.

(III) The cationic polyvinyllactams described in the French Patent Application Publication No. FR-A-2 820 032. These polymers comprise:
(a) at least one vinyllactam or alkylvinyllactam monomer;
(b) at least one monomer chosen from structure (I) and (II): 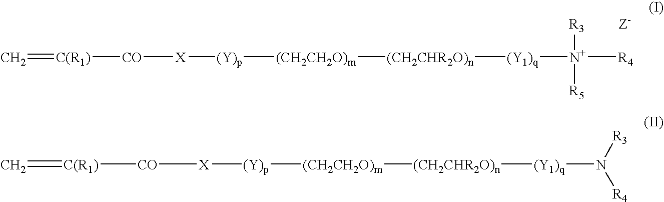
   wherein:

X is chosen from an oxygen atom or NR₆ radicals,

R₁ and R₆, which may be identical or different, are chosen from hydrogen atoms and linear and branched C₁-C₅ alkyl radicals,

R₂ is chosen from linear and branched C₁-C₄ alkyl radicals,

R₃, R₄ and R₅, which may be identical or different, are chosen from hydrogen atoms, linear and branched C₁-C₃₀ alkyl radicals, and radicals of formula (III):
—(Y₂)ᵣ—(CH₂—CH(R₇)—O)ₓ—R₈ (III)
wherein

Y, Y₁ and Y₂, which may be identical or different, are chosen from linear and branched C₂-C₁₆ alkylene radicals,

R₇ is chosen from a hydrogen atom, linear and branched C₁-C₄ alkyl radicals, and linear and branched C₁-C₄ hydroxyalkyl radicals,

R₈ is chosen from a hydrogen atom and linear and branched C₁-C₃₀ alkyl radicals,

p, q and r, which may be identical or different, are chosen from the value zero and the value 1,

m and n, which may be identical or different, are integers ranging from 0 to 100,

x is an integer ranging from 1 to 100, and

Z is an anion of an organic or inorganic acid,

with the provisos that:

at least one of the substituents, R₃, R₄, R₅ or R₈, is chosen from linear and branched C₉-C₃₀ alkyl radicals,

if m or n is other than zero, q is 1, and

if m or n are zero, p or q is 0.

These cationic poly(vinyllactam) polymers may be crosslinked or non-crosslinked and may be random or block. For example, in one embodiment, R₃, R₄ and R₅, which may be identical or different, are chosen from hydrogen atoms and linear and branched C₁-C₃₀ alkyl radicals. In another embodiment, the monomer (b) is a monomer of formula (I) for which m and n are zero.

The vinyllactam or alkylvinyllactam may be, for example, a compound chosen from those of formula (IV): 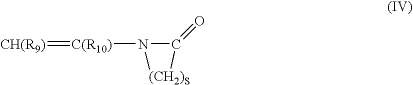
wherein:

s is an integer ranging from 3 to 6,

R₉ is chosen from a hydrogen atom and C₁-C₅ alkyl radicals, and

R₁₀ is chosen from a hydrogen atom and C₁-C₅ alkyl radicals,

with the proviso that at least one of the radicals R₉ and R₁₀ is a hydrogen atom.

For example, in one embodiment, the monomer (IV) is vinylpyrrolidone.

The associative cationic poly(vinyllactams) above may also comprise at least one additional monomer, such as cationic and nonionic monomers.

Cationic polyvinyllactams that may be used include, by way of non-limiting example, the following terpolymers comprising:

(a)—at least one monomer of formula (IV),

(b)—at least one monomer of formula (I) in which p is equal to 1, m and n are both zero, q is equal to zero, R₃ and R₄, which may be identical or different, are chosen from hydrogen atoms and C₁-C₅ alkyl radicals, and R₅ is chosen from C₉-C₂₄ alkyl radicals, and

(c)—at least one monomer of formula (II) in which R₃ and R₄, which may be identical or different, are chosen from hydrogen atoms and C₁-C₅ alkyl radicals.

In one embodiment, use is made of terpolymers comprising, by weight, from 40% to 95% of monomer (a), from 0.1% to 55% of monomer (c) and from 0.25% to 50% of monomer (b).

Polymers of this kind are described in International Patent Application Publication No. WO 00/68282.

Among the cationic poly(vinyllactam) polymers that may be used according to the present disclosure, mention may be made, for example, of vinylpyrrolidone/dimethylaminopropylmethacrylamide/dodecyldimethylmethacrylamidopropylammonium tosylate terpolymers, vinylpyrrolidone/dimethylaminopropylmethacrylamide/cocoyldimethylmethacrylamidopropylammonium tosylate terpolymers and vinylpyrrolidone/dimethylaminopropylmethacrylamide/lauryldimethylmethacrylamidopropylammonium chloride and/or tosylate terpolymers.

In one embodiment of the present disclosure, the amphoteric associative polymers can be chosen from those comprising at least one non-cyclic cationic unit, for instance, those comprising from 1 mol % to 20 mol % of at least one monomer comprising a fatty chain.

In another embodiment, the amphoteric associated polymers according to the present disclosure can comprise, or can be prepared by copolymerizing:

1) at least one monomer chosen from those of formula (Ia) (Ib):

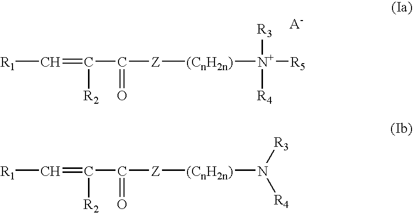

wherein R₁ and R₂, which may be identical or different, are chosen from hydrogen atoms and methyl radicals, R₃, R₄ and R₅, which may be identical or different, are chosen from linear and branched alkyl radicals comprising from 1 to 30 carbon atoms,

Z is chosen from NH groups and oxygen atoms,

n is an integer from 2 to 5, and

A⁻ is chosen from anions obtained from organic and inorganic acids, such as a methosulphate anion, halides, such as chloride or bromide;

2) at least one monomer chosen from those of formula (II):
R₆—CH═CR₇—COOH (II)
wherein R₆ and R₇, which may be identical or different, are chosen from hydrogen atoms and methyl radicals, and

3) at least one monomer chosen from those of formula (III):
R₆—CH═CR₇—COXR₈ (III)

wherein R₆ and R₇, which may be identical or different, are chosen from hydrogen atoms and methyl radicals, X is chosen from oxygen and nitrogen atoms and R₈ is chosen from linear and branched alkyl radicals comprising from 1 to 30 carbon atoms;

wherein at least one of the monomers of formula (Ia), (Ib) and (III) comprises at least one fatty chain.

The monomers of formula (Ia) and (Ib) of the present disclosure can be chosen from, for example, the group comprising dimethylaminoethyl methacrylate, dimethylaminoethyl acrylate, diethylaminoethyl methacrylate, diethylaminoethyl acrylate, dimethylaminopropyl methacrylate, dimethylaminopropyl acrylate, dimethylaminopropylmethacrylamide, dimethylaminopropylacrylamide, these monomers being optionally quaternized, for example by a C₁-C₄ alkyl halide or a di-C₁-C₄ alkyl sulphate.

In one embodiment of the present disclosure, the at least one monomer of formula (Ia) is chosen from acrylamidopropyltrimethylammonium chloride and methacrylamidopropyltrimethylammonium chloride.

In another embodiment of the present disclosure, the at least one monomer of formula (II) is chosen from acrylic acid, methacrylic acid, crotonic acid and 2-methylcrotonic acid. For example, In one embodiment the at least monomer of formula (II) is acrylic acid.

In yet another embodiment of the present disclosure, the at least one monomer of formula (III) is chosen from C₁₂-C₂₂, such as C₁₆-C₁₈, alkyl acrylates and methacrylates.

In the amphoteric associative polymers of the present disclosure, the numerical cationic charge/anionic charge ratio can be, in one embodiment, equal to 1.

These amphoteric associative polymers and their preparation are described for example, in International Patent Application Publication No. WO 98/44012.

Among these amphoteric associative polymers, mention may be made of acrylic acid/(meth)acrylamidopropyltrimethylammonium chloride/stearyl methacrylate terpolymers.

Finally, the nonionic associative polymers used in the present disclosure can be, for example, chosen from:

(1) celluloses, for instance hydroxyethylcelluloses, modified with groups comprising at least one fatty chain. Non-limiting examples include:

hydroxyethylcelluloses modified by groups comprising at least one fatty chain, such as alkyl, arylalkyl, alkylaryl groups, and mixtures thereof, and in which the alkyl groups can be, for instance, C₈-C₂₂, such as the product Natrosol Plus Grade 330 CS® (C₁₆ alkyl), sold by Aqualon, or the product Bermocoll EHM 100®, sold by Berol Nobel,

hydroxyethylcelluloses modified with alkylphenol polyalkylene glycol ether groups, such as the product Amercell Polymer HM-1500® (nonylphenol polyethylene glycol (15) ether), sold by Amerchol.

(2) hydroxypropylguars modified with groups comprising at least one fatty chain, such as the product Esaflor HM 22® (C₂₂ alkyl chain), sold by Lamberti, and the products RE210-18® (C₁₄ alkyl chain) and RE205-1® (C₂₀ alkyl chain), sold by Rhône Poulenc.

(3) copolymers of vinylpyrrolidone and fatty-chain hydrophobic monomers; non-limiting examples include:

the products Antaron V216® or Ganex V216® (vinylpyrrolidone/hexadecene copolymer), sold by I.S.P.,

the products Antaron V220® or Ganex V220® (vinylpyrrolidone/eicosene copolymer) sold by I.S.P.

(4) copolymers of C₁-C₆ alkyl acrylates or methacrylates and amphiphilic monomers comprising at least one fatty chain, such as, for example, the methyl acrylate/ethoxylated stearyl acrylate copolymer sold by Goldschmidt under the name Antil 208®.

(5) copolymers of hydrophilic acrylates or methacrylates and hydrophobic monomers comprising at least one fatty chain, such as, for example, polyethylene glycol methacrylate/lauryl methacrylate copolymer.

(6) polyether polyurethanes comprising in their chain not only hydrophilic blocks, usually of polyethoxylated type, but also hydrophobic blocks, which may be aliphatic concatenations alone and/or cycloaliphatic and/or aromatic concatenations.

In one embodiment of the present disclosure, the polyether-polyurethanes comprise at least two lipophilic hydrocarbon chains comprising from 6 to 30 carbon atoms, separated by a hydrophilic block, it being possible for the hydrocarbon chains to be pendant chains or chains at the end of the hydrophilic block. For instance, it is possible for at least one pendant chain to be provided. For further example, the polymer may include a hydrocarbon chain at one end or at both ends of a hydrophilic block.

The polyether-polyurethanes can be multiblock polymers, for instance, in triblock form. The hydrophobic blocks may be at each end of the chain (for example: triblock copolymer with hydrophilic middle block) or may be distributed both at the ends and in the chain (multiblock copolymer, for example). These same polymers can also be graft polymers or star polymers.

The nonionic fatty-chain polyether-polyurethanes can be triblock copolymers whose hydrophilic block is a polyethoxylated chain comprising from 50 to 1000 oxyethylene groups. The nonionic polyurethane polyethers comprise a urethane linkage between the hydrophilic blocks, hence the name.

In addition, the fatty-chain nonionic polyether-polyurethanes can also include those whose hydrophilic blocks are linked to the lipophilic blocks via other chemical linkages.

As non-limiting examples of fatty-chain nonionic polyether-polyurethanes which can be used in the present disclosure, it is also possible to use the urea-functional Rheolate 205®, sold by Rheox, or else Rheolates® 208, 204 or 212, and also Acrysol RM 184®.

Non-limiting mention may also be made of the product Elfacos T210®, containing a C₁₂₋₁₄ alkyl chain, and of the product Elfacos T212® containing a C₁₈ alkyl chain, from Akzo.

The product DW 1206B® from Rohm & Haas, having a C₂₀ alkyl chain and a urethane linkage, provided at 20% solids in water, may also be mentioned in a non-limiting manner.

It is also possible to use solutions or dispersions of these polymers, for instance, in water or in an aqueous-alcoholic medium. Non-limiting examples of such polymers include Rheolate® 255, Rheolate® 278 and Rheolate® 244, sold by Rheox. It is also possible to use the products DW 1206F and DW 1206J, which are provided by Rohm & Haas.

The polyether-polyurethanes useful according to the present disclosure also include those described in the article by G. Fonnum, J. Bakke and Fk. Hansen—Colloid Polym. Sci. 271, 380.389 (1993).

In one embodiment, according to the present disclosure, use is made of a polyether-polyurethane obtainable by polycondensing at least three compounds including (i) at least one polyethylene glycol comprising 150 mol to 180 mol of ethylene oxide, (ii) stearyl alcohol or decyl alcohol and (iii) at least one diisocyanate.

Polyether polyurethanes of this kind are sold, for example, by Rohm & Haas under the names Aculyn 46® and Aculyn 44®. Aculyn 46® is a polycondensate of polyethylene glycol containing 150 or 180 mol of ethylene oxide, stearyl alcohol and methylenebis(4-cyclohexyl isocyanate) (SMDI), at 15% by weight in a matrix of maltodextrin (4%) and water (81%). Aculyn 44® is a polycondensate of polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%).

(7) copolymers of PEG-180, tetramethoxymethyl glycoluril and laureth-50 or octoxynol-40 (INCI names), provided under the names Pure Thix® 1450 and Pure Thix® 1451 by Sud-Chemie.

In one embodiment, the associative polymers used according to the present disclosure possess at least two C₈-C₄₀ fatty chains.

In another embodiment, the associative polymers used in the cosmetic compositions of the present disclosure in combination with the diblock polymers are associative cationic polyurethanes of formula (Ia), described in detail above.

As shown by the example below, these polymers, when used, for instance, in combination with anionic diblock block copolymers for thickening aqueous media, can give very high thickening factors.

The aqueous cosmetic compositions of the present disclosure, thickened by the combination of at least one associative polymer and at least one amphiphilic diblock copolymer, may be compositions for washing keratin materials, such as liquid soaps and shampoos, conditioners, direct or oxidation dyeing compositions for hair, reducing compositions for the permanent shaping of hair or for the stripping of color, oxidizing compositions for the coloring or bleaching of hair, alkaline compositions for straightening, and temporary hair shaping compositions.

These compositions may also be compositions for the treatment of the skin and mucosae, such as anti-sun compositions, lip makeup compositions and/or skin makeup compositions.

The compositions may also be lash makeup compositions, such as mascaras.

In one embodiment of the present disclosure, the compositions of the present disclosure are hair compositions.

The compositions as disclosed herein may further comprise, at least one commonly used formulating additive or cosmetic active principle, such as anionic, nonionic, cationic and amphoteric film-forming polymers; natural and synthetic polymeric thickeners other than the associative polymers and amphiphilic diblock copolymers described above; non-polymeric thickeners such as acids or electrolytes; nonionic, anionic, cationic and zwitterionic surfactants; pearlizing agents; opacifiers; dyes; pigments; perfumes; mineral, vegetable and/or synthetic oils; waxes; ceramides; vitamins; UV screening agents; anti-dandruff agents; free-radical scavengers; plasticizers; preservatives; and pH stabilizers.

The skilled artisan will select any additives and their amount such the beneficial properties of the compositions of the present disclosure will not be substantially lessed, namely the thickening effect of the polymer combination claimed.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following example illustrates the present disclosure in a non-limiting manner.

### EXAMPLE

### Thickening of an Aqueous Medium by the Combination of a Cationic Associative Polyurethane and an Anionic Linear Diblock Block Copolymer—Demonstration of Synergistic Effect

An aqueous solution was prepared comprising 2.5% by weight of active substance of a cationic polyethoxylated polyurethane, prepared from PEG-10,000, dicyclohexylmethane diisocyanate, and 2-dimethylaminoethanol, and quaternized to the extent of 100% by 1-bromododecane (polyurethane X) or bromooctadecane (polyurethane Y).

The viscosity of this solution was measured by means of a Haake RS 600 rheometer at a temperature of 25.0° C. and a shear rate of 1 s⁻¹. Added to this aqueous solution of associative polymer were increasing amounts of a linear diblock block copolymer A (polystyrene-poly(sodium acrylate)), and the viscosity of the solution thus obtained was measured under the same conditions as before.

The plots exhibit a maximum at a diblock copolymer concentration of 0.25%. The existence of this maximum, even when it is only a local maximum, shows a thickening synergy, where the maximum viscosity corresponds to the stoichiometric ratio permitting optimum interaction between the associative polymer and the diblock copolymer.

Thickening Factor

From each “bell curve” such as that shown in FIG. 1 attached it is possible to calculate the thickening factor, which is defined as the ratio between the viscosity at the (local) maximum of the plot and the viscosity of the solution of associative polymer alone at the concentration used.

The table below shows the thickening factors of different associative polymer/diblock copolymer pairings. This table shows that polyurethanes X and Y allowed high thickening factors to be obtained.

| | | Viscosity | | Concentration | | |
|---|---|---|---|---|---|---|
| | Concentration | at 1 s⁻¹ | | of diblock | Viscosity of | |
| Associative | (% by | and 25° C. | Diblock | polymer | mixture at | Thickening |
| polymer | weight)^{(a)} | (Pa · s) | polymer | (% by weight) | 1 s⁻¹(Pa · s)^{(b)} | factor |
| Polyurethane Y | 2.5 | 1.0 | A | 0.25 | 71 | 71 |
| Polyurethane X | 2.5 | 0.008 | A | 0.25 | 0.22 | 27.5 |

^{(a)}mass fraction of active substance
^{(b)}values measured after 30 seconds of shearing with a Haake RS 600 rheometer operating with applied stress in cone/plate geometry, diameter 60 mm/1° (titanium), at a temperature regulated at 25.0° C. by a Peltier-effect plate, the device as a whole being situated under a solvent bell intended to prevent the evaporation of the solvent.
Diblock polymer A: Polystyrene-poly(sodium acrylate), number-average molar mass 4,500 (polystyrene block) and 30,000 (poly(sodium acrylate) block), sold by Polymer Source.

What is claimed is:

1. A cosmetic composition comprising, in a physiologically acceptable aqueous medium, a combination of
at least one associative polymer comprising at least one hydrophilic part and at least one C₈₋₄₀ fatty chain, and
at least one linear diblock block copolymer comprising a hydrophobic block and a hydrophilic block,
with the proviso that the linear diblock copolymer does not comprise block copolymers of ethylene oxide and propylene oxide, block copolymers with urethane units, and block copolymers with siloxane units.

2. The cosmetic composition according to claim 1, wherein the at least one linear diblock copolymer is chosen from anionic and nonionic linear diblock block copolymers.

3. The cosmetic composition according to claim 1, wherein the at least one associative polymer comprising at least one hydrophilic part and at least one C₈₋₄₀ fatty chain is soluble in the aqueous medium.

4. The cosmetic composition according to claim 1, wherein the at least one associative polymer comprising at least one hydrophilic part and at least one C₈₋₄₀ fatty chain is chosen from cationic, anionic and nonionic associative polymers.

5. The cosmetic composition according to claim 4, wherein the at least one cationic associative polymer is chosen from cationic associative polyurethanes; quaternized celluloses comprising fatty chains chosen from C₈₋₃₀ alkyl, arylalkyl and/or alkylaryl groups; quaternized hydroxyethylcelluloses comprising fatty chains chosen from C₈₋₃₀ alkyl, arylalkyl and/or alkylaryl groups; and cationic polyvinyllactams.

6. The cosmetic composition according to claim 4, wherein the at least one anionic associative polymer is chosen from copolymers of (meth)acrylic acid and C₈₋₃₀ fatty alcohol allyl ethers; copolymers of unsaturated carboxylic acids and C₁₀₋₃₀ alkyl unsaturated carboxylates; terpolymers of maleic anhydride/C₃₀₋₃₈ α-olefin/alkyl maleate; acrylic terpolymers of an α,β-unsaturated carboxylic acid, a non-surfactant α,β-unsaturated monomer different from said acid, and a nonionic surfactant monomer obtained by reacting an ethylenically unsaturated monoisocyanate and a monohydric surfactant; and copolymers of α,β-unsaturated carboxylic acids and alkoxylated fatty alcohol carboxylates.

7. The cosmetic composition according to claim 4, wherein the at least one nonionic associative polymer is chosen from celluloses modified with groups comprising at least one fatty chain; hydroxypropylguars modified with groups comprising at least one fatty chain; copolymers of vinylpyrrolidone and fatty-chain hydrophobic monomers; copolymers of C₁₋₆ alkyl (meth)acrylates and amphiphilic monomers comprising at least one fatty chain; copolymers of hydrophilic (meth)acrylates and hydrophobic monomers comprising at least one fatty chain; polyether-polyurethanes comprising hydrophilic blocks and at least one fatty chain; and copolymers of PEG-180, tetramethoxymethylglycoluril and laureth-50 or octoxynol-40.

8. The cosmetic composition according to claim 5, wherein the at least one cationic associative polymer is chosen from cationic associative polyurethanes of formula (Ia):
R—X—(P)ₙ—[L—(Y)ₘ]ᵣ—L′—(P′)ₚ—X′—R′ (Ia)
wherein:
R and R′, which may be identical or different, are chosen from hydrophobic groups and hydrogen atoms;
X and X′, which may be identical or different, are chosen from groups comprising an amine functional group optionally carrying a hydrophobic group; and L″ groups;
L, L′ and L″, which may be identical or different, are chosen from groups derived from diisocyanates;
P and P′, which may be identical or different, are chosen from groups comprising an amine functional group optionally carrying a hydrophobic group;
Y is chosen from hydrophilic groups;
r is an integer ranging from 1 to 100,
n, m and p each independently of one another range from 0 to 1,000, and
wherein the polyurethane of formula (Ia) comprises at least one functional group chosen from protonated and quaternized amine functional groups and at least one hydrophobic group.

9. The cosmetic composition according to claim 8, wherein r is an integer ranging from 1 to 25.

10. The cosmetic composition according to claim 8, wherein the at least one cationic polyurethane is a PEG 10,000/dicyclohexylmethane diisocyanate/2-dimethylaminoethanol copolymer quaternized with 1-bromododecane or 1-bromooctadecane.

11. The cosmetic composition according to claim 1, wherein the at least one linear diblock block copolymer composed of a hydrophobic block and a hydrophilic block is chosen from anionic polymers.

12. The cosmetic composition according to claim 1, wherein the at least one linear diblock block copolymer is soluble in the aqueous medium.

13. The cosmetic composition according to claim 1, wherein the hydrophilic block of the at least one linear diblock block copolymer is formed from at least one anionic water-soluble monomer or from a mixture of at least one anionic and at least one nonionic water-soluble monomer.

14. The cosmetic composition according to claim 13, wherein the at least one anionic water-soluble monomer is chosen from ethylenically unsaturated carboxylic acids, 2-acrylamido-2-methylpropanesulphonic acid, styrenesulphonic acid, vinylsulphonic acid, vinylphosphonic acid, and the salts thereof.

15. The cosmetic composition according to claim 13, wherein the at least one nonionic water-soluble monomer is chosen from acrylamide, N—C₁₋₆ alkyl-acrylamides, N,N-di-C₁₋₃alkyl-acrylamides, polyethylene glycol acrylate, polyethylene glycol methacrylate, N-vinylacetamide, N-methyl-N-vinylacetamide, N-vinylformamide, N-methyl-N-vinylformamide, N-vinyl lactams comprising a cyclic group of 4 to 9 carbon atoms, vinyl alcohol, ethylene oxide, hydroxyethyl acrylate, hydroxypropyl acrylate, hydroxyethyl methacrylate and hydroxypropyl methacrylate.

16. The cosmetic composition according to claim 1, wherein the hydrophobic block of the at least one linear diblock block copolymer is formed from at least one water-insoluble monomer chosen from vinylaromatic monomers; dienes; alkyl derivatives of dienes; chloroprene; C₁₋₁₀ alkyl acrylates; C₆₋₁₀ aryl acrylates; C₁₋₁₀ aralkyl acrylates; C₁₋₁₀ alkyl methacrylates; C₆₋₁₀ aryl methacrylates; C₁₋₁₀ aralkyl methacrylates; vinyl acetate; vinyl ethers chosen from those of formula CH₂═CH—O—R and allyl ethers chosen from those of formula CH₂═CH—CH₂—O—R wherein R is chosen from C₁₋₆ alkyl groups; acrylonitrile; vinyl chloride; vinylidene chloride; caprolactone; ethylene; propylene; fluorinated vinyl monomers; and vinyl monomers comprising a perfluorinated chain.

17. The cosmetic composition according to claim 1, wherein the hydrophilic block of the at least one linear diblock block copolymer comprises less than or equal to 25 mol % of at least one water-insoluble monomer chosen from vinylaromatic monomers; dienes; alkyl derivatives of dienes; chloroprene; C₁₋₁₀ alkyl acrylates; C₆₋₁₀ aryl acrylates; C₁₋₁₀ aralkyl acrylates; C₁₋₁₀ alkyl methacrylates; C₆₋₁₀ aryl methacrylates; C₁₋₁₀ aralkyl methacrylates; vinyl acetate; vinyl ethers chosen from those of formula CH₂═CH—O—R and allyl ethers chosen from those of formula CH₂═CH—CH₂—O—R wherein R is chosen from C₁₋₆ alkyl groups; acrylonitrile; vinyl chloride; vinylidene chloride; caprolactone; ethylene; propylene; fluorinated vinyl monomers; and vinyl monomers comprising a perfluorinated chain.

18. The cosmetic composition according to claim 17, wherein the hydrophilic block of the at least one linear diblock block copolymer comprises less than or equal to 10 mol % of the at least one water-insoluble monomer chosen from vinylaromatic monomers; dienes; alkyl derivatives of dienes; chloroprene; C₁₋₁₀ alkyl acrylates; C₆₋₁₀ aryl acrylates; C₁₋₁₀ aralkyl acrylates; C₁₋₁₀ alkyl methacrylates; C₆₋₁₀ aryl methacrylates; C₁₋₁₀ aralkyl methacrylates; vinyl acetate; vinyl ethers chosen from those of formula CH₂═CH—O—R and allyl ethers chosen from those of formula CH₂═CH—CH₂—O—R wherein R is chosen from C₁₋₆ alkyl groups; acrylonitrile; vinyl chloride; vinylidene chloride; caprolactone; ethylene; propylene; fluorinated vinyl monomers; and vinyl monomers comprising a perfluorinated chain.

19. The cosmetic composition according to claim 18, wherein the hydrophilic block of the at least one linear diblock block copolymer comprises less than or equal to 5 mol % of the at least one water-insoluble monomer chosen from vinylaromatic monomers; dienes; alkyl derivatives of dienes; chloroprene; C₁₋₁₀ alkyl acrylates; C₆₋₁₀ aryl acrylates; C₁₋₁₀ aralkyl acrylates; C₁₋₁₀ alkyl methacrylates; C₆₋₁₀ aryl methacrylates; C₁₋₁₀ aralkyl methacrylates; vinyl acetate; vinyl ethers chosen from those of formula CH₂═CH—O—R and allyl ethers chosen from those of formula CH₂═CH—CH₂—O—R wherein R is chosen from C₁₋₆ alkyl groups; acrylonitrile; vinyl chloride; vinylidene chloride; caprolactone; ethylene; propylene; fluorinated vinyl monomers; and vinyl monomers comprising a perfluorinated chain.

20. The cosmetic composition according to claim 1, wherein the hydrophobic block of the at least one linear diblock block copolymer comprises less than or equal to 25 mol % of at least one water-soluble monomer chosen from acrylamide, N—C₁₋₆ alkyl-acrylamides, N,N-di-C₁₋₃ alkyl-acrylamides, polyethylene glycol acrylate, polyethylene glycol methacrylate, N-vinylacetamide, N-methyl-N-vinylacetamide, N-vinylformamide, N-methyl-N-vinylformamide, N-vinyl lactams comprising a cyclic group of 4 to 9 carbon atoms, vinyl alcohol, ethylene oxide, hydroxyethyl acrylate, hydroxypropyl acrylate, hydroxyethyl methacrylate, hydroxypropyl methacrylate, ethylenically unsaturated carboxylic acids, 2-acrylamido-2-methylpropanesulphonic acid, styrenesulphonic acid, vinylsulphonic acid, vinylphosphonic acid, and the salts thereof.

21. The cosmetic composition according to claim 20, wherein the hydrophobic block of the at least one linear diblock block copolymer comprises less than or equal to 10 mol % of at least one water-soluble monomer chosen from acrylamide, N—C₁₋₆ alkyl-acrylamides, N,N-di-C₁₋₃ alkyl-acrylamides, polyethylene glycol acrylate, polyethylene glycol methacrylate, N-vinylacetamide, N-methyl-N-vinylacetamide, N-vinylformamide, N-methyl-N-vinylformamide, N-vinyl lactams comprising a cyclic group of 4 to 9 carbon atoms, vinyl alcohol, ethylene oxide, hydroxyethyl acrylate, hydroxypropyl acrylate, hydroxyethyl methacrylate, hydroxypropyl methacrylate, ethylenically unsaturated carboxylic acids, 2-acrylamido-2-methylpropanesulphonic acid, styrenesulphonic acid, vinylsulphonic acid, vinylphosphonic acid, and the salts thereof.

22. The cosmetic composition according to claim 21, wherein the hydrophobic block of the at least one linear diblock block copolymer comprises less than or equal to 5 mol % of at least one water-soluble monomer chosen from acrylamide, N—C₁₋₆ alkyl-acrylamides, N,N-di-C₁₋₃ alkyl-acrylamides, polyethylene glycol acrylate, polyethylene glycol methacrylate, N-vinylacetamide, N-methyl-N-vinylacetamide, N-vinylformamide, N-methyl-N-vinylformamide, N-vinyl lactams comprising a cyclic group of 4 to 9 carbon atoms, vinyl alcohol, ethylene oxide, hydroxyethyl acrylate, hydroxypropyl acrylate, hydroxyethyl methacrylate, hydroxypropyl methacrylate, ethylenically unsaturated carboxylic acids, 2-acrylamido-2-methylpropanesulphonic acid, styrenesulphonic acid, vinylsulphonic acid, vinylphosphonic acid, and the salts thereof.

23. The cosmetic composition according to claim 1, wherein the at least one linear diblock block copolymer comprises a hydrophobic homopolymer block and an anionic hydrophilic homopolymer block.

24. The cosmetic composition according to claim 23, wherein the hydrophobic homopolymer block is polystyrene and the anionic hydrophilic homopolymer block is chosen from poly(sodium acrylate) and poly(sodium styrenesulphonate).

25. The cosmetic composition according to claim 1, wherein the ratio by weight of the hydrophobic block to the hydrophilic block of the at least one diblock copolymer ranges from 1/20 to 20/1.

26. The cosmetic composition according to claim 25, wherein the ratio by weight of the hydrophobic block to the hydrophilic block of the at least one diblock copolymer ranges from 1/10 to 10/1.

27. The cosmetic composition according to claim 1, wherein the at least one associative polymer is present in an amount ranging from 0.01% to 20% by weight, relative to the total weight of the composition.

28. The cosmetic composition according to claim 27, wherein the at least one associative polymer is present in an amount ranging from 0.05% to 10% by weight, relative to the total weight of the composition.

29. The cosmetic composition according to claim 28, wherein the at least one associative polymer is present in an amount ranging from 0.1% to 5% by weight, relative to the total weight of the composition.

30. The cosmetic composition according to claim 1, wherein the at least one linear diblock block copolymer is present in an amount ranging from 0.001% to 20% by weight, relative to the total weight of the composition.

31. The cosmetic composition according to claim 30, wherein the at least one linear diblock block copolymer is present in an amount ranging from 0.005% to 10% by weight, relative to the total weight of the composition.

32. The cosmetic composition according to claim 31, wherein the at least one linear diblock block copolymer is present in an amount ranging from 0.01% to 5% by weight, relative to the total weight of the composition.

33. The cosmetic composition according to claim 1, wherein the weight ratio of the at least one associative polymer to the at least one diblock polymer ranges from 0.01 to 100.

34. The cosmetic composition according to claim 33, wherein the weight ratio of the at least one associative polymer to the at least one diblock polymer ranges from 1 to 50.

35. The cosmetic composition according to claim 34, wherein the weight ratio of the at least one associative polymer to the at least one diblock polymer ranges from 5 to 30.

36. A method for thickening or gelling an aqueous cosmetic composition comprising adding to the composition a combination of
at least one associative polymer comprising at least one hydrophilic part and at least one C₈₋₄₀ fatty chain, and
at least one linear diblock block copolymer composed of a hydrophobic block and a hydrophilic block,
with the proviso that the at least one linear diblock block copolymer is not chosen from block copolymers of ethylene oxide and propylene oxide, block copolymers with urethane units, and block copolymers with siloxane units.

37. The method according to claim 36, wherein the at least one linear diblock block copolymer is chosen from anionic and nonionic linear diblock block copolymers.

38. The method according to claim 36, wherein the aqueous cosmetic composition is chosen from aqueous hair treatment compositions.
